(19) United States
(12) Patent Application Publication
(10) Pub. No.: US 2008/0050320 A1
(43) Pub. Date: Feb. 28, 2008

(54) SKIN CARE COMPOSITIONS CONTAINING A HYDROPHOBIC MATERIAL AND RELATED METHODS

(76) Inventors: Ariel Haskel, East Brunswick, NJ (US); Paloma Pimenta, Staten Island, NY (US)

Correspondence Address: COLGATE-PALMOLIVE COMPANY, 909 RIVER ROAD, PISCATAWAY, NJ, 08855, US

(21) Appl. No.: 11/683,801

(22) Filed: Mar. 8, 2007

Related U.S. Application Data
(60) Provisional application No. 60/823,305, filed on Aug. 23, 2006.

(51) Int. Cl. A61K 47/32; A61Q 17/04

(52) U.S. Cl. 424/59; 514/772.6

(57) ABSTRACT

The invention includes a skin care composition comprising an acrylate polymer; a yield value-increasing polymer; and a non-silicone hydrophobic material. The composition has a yield value of at least about 4. Also included within the scope of the invention are related methods, such as methods of depositing a hydrophobic material on an epidermal surface and methods of incorporating a hydrophobic material into a skin care composition.

### CROSS REFERENCE TO RELATED APPLICATION

This application claims the benefit of U.S. Provisional Patent Application Ser. No. 60/823,303 filed Aug. 23, 2006, the contents of which are incorporated herein by reference.

### BACKGROUND OF THE INVENTION

Moisturizing formulations for skin care typically contain hydrophobic materials such as petrolatum or other oils or waxes which include liquids, solid or semi-solid at room temperature, in the form of oil-in-water emulsions. The hydrophobic materials are typically introduced into the aqueous medium by adding under shear stress a hot oil phase containing, e.g., petrolatum, into a hot aqueous phase in the presence of emulsifiers to allow for homogenous dispersion of the petrolatum into the surfactant system. Simply incorporating melted petrolatum or similar material into a cold base is not feasible as the melted petrolatum solidifies upon contact with the base material. The need for hot processing, however, renders the manufacturing relatively expensive and difficult. Moreover, the emulsifiers can interfere with the desired deposition of the hydrophobic agents on the skin.

Accordingly, there is a need in the art for a skin care composition that meets the consumer's requirements of providing moisturizing skin benefits and deposition of moisturizing materials, and which can be processed at ambient or close to ambient temperatures.

### BRIEF SUMMARY OF THE INVENTION

The invention includes a skin care composition comprising an acrylate polymer; a yield value-increasing polymer; and a non-silicone hydrophobic material. The composition has a yield value of at least about 4. Also included within the scope of the invention are related methods, such as methods of depositing a hydrophobic material on an epidermal surface and methods of incorporating a hydrophobic material into a skin care composition.

Methods of depositing a hydrophobic material on an epidermal surface, include, for example, the steps of: (a) applying to the epidermal surface a composition comprising skin care composition comprising: an acrylate polymer; a modified guar gum; and a hydrophobic material, wherein the composition has a yield stress value of at least about 4; and (b) removing the composition from the epidermal surface, wherein at least a portion of the hydrophobic material remains deposition on the surface.

Methods of incorporating a hydrophobic material into a skin care composition, include, for example, the steps of: (a) forming a base, the base comprising an acrylate polymer; and (b) combining the base with a hydrophobic material and a cationic yield value-increasing polymer. This method may be carried out at a temperature of no greater than 40° C.

### DETAILED DESCRIPTION OF THE INVENTION

Described herein are skin care compositions that may be prepared by cold processes and include at least an acrylate polymer, a yield increasing polymer, and a hydrophobic material. Preferably, the composition has a yield value of at least about 4.

The skin care composition may be a substantially non-emulsified composition. The term “non-emulsified” as used herein means a composition other than an emulsion, or a composition which is not an O/W type emulsion or a W/O type emulsion. In other words, when two solutions are mixed one phase is not dispersed in the other phase in the form of micelles that are completely encapsulated by an emulsifying agent to effectively separate one phase from the other.

By “cold process” it is meant that at least the step of incorporating the hydrophobic material into the composition or into a pre-mix intended to form part of the final composition may be carried out at ambient temperature, for example, about 10° C. to about 100° C., preferably about 15° C. to about 30° C., and/or up to about 40° C.

The composition includes in acrylate homopolymer or copolymer (hereinafter collectively referred to as “polymer”). The acrylate polymer may be any known or to be developed in the art and may include swellable acrylate co-polymer, e.g., such as CARBOPOL® Aqua SF-1 (from Noveon, Cleveland, Ohio, United States of America). Suitable polymers (including homopolymers or co-polymers of the listed components) may include those of 2-hydroxyethyl acrylate, hydroxypropyl acrylate, polymers of acrylic acid and its esters, polymers of methacrylic acid and its esters, acrylnitriles, ethylacrylate, methacrylate, polyalkenyl ethers of sucrose, polyalkenyl ethers of polyalcohols, trimethylpropone tri(meth)acrylate, glycidal methacrylate, and N-methylolacryamide. Other polymers that may be suitable are described in U.S. Pat. No. 6,635,702, the contents of which are incorporated herein by reference. Mixtures of polymers (co- and homo-) may also be used, if desired.

The selected polymer(s) may be present in the composition in any amount. It may be preferred that they are present in an amount of about 1% to about 30% by weight of the total skin care composition, with amounts of about 3% to about 12%, and about 5% to 12% of the total weight of the skin care composition also being suitable.

The composition of the invention includes a yield-increasing polymer that is different from the acrylate polymer. By “yield-increasing polymer” (“YIP”) it is meant a polyymier that, when added to an acrylate polymer and surfactant rnixture the yield value increased by about at least 5%, about 10%, about 15%, about 20%, about 25%, about 30%, about 40% or more (yield values measured as described herein).

The yield-increasing polymer may be a cationic polymer. It may be, for example, a cationic gum. Preferably the gum is a salt of guar hydroxypropyltrimolilun (such as guar hydroxypropyltrimonium chloride) or other cationic gum.

The YIP may be present in the composition in anv amount. Preferably, it is present in an amount of up to about 20% be weight. Alternatively, amounts may be up to about 10% bv weight, about 0.1% to about 5% by weight and about 7% to about 12% bv weight.

The skin care composition may also include a hydrophobic material, winch may be a non-silicone hydrophobic material. Such material may be liquid, solid or semi-solid at room temperature and mav function to confer a skin benefit when applied topically, e.g., skin moisturization. Exemplary hydrophobic materials include: mineral oils, synthetic oils, vegetable oils, semi-solid hydrocarbons, petrolatum, long chain alkanes and alkenes, isodedecane, isohexadecane, hydrogenated polydecene, polydecene, and hydrocarbons found in beeswax (for example, C₂₁₋₃₇ hydrocarbons), esters of fatty acids (including hydrogenated forms or derivatives thereof) and long straight chain alcohols, triacontanol hexadecanoate, hexacosanol hexacosanoate, and myricyl palmitate, fatty acid triglycerides, animal fats, lanolin, hydrogenated or partially hydrogenated vegetable oils, hydrogenated or partially hydrogenated sunflower oil, safflower oil, soybean oil, rapeseed oil, grape seed oil, corn oil, olive oil, sweet almond oil, coconut oil, palm kernel oil, soybean oil, and/or derivatives thereof, castoryl maleate, hydrophobic vegetable extracts, shea butter, cocoa butter, and derivatives and fractions thereof; hydrophobic UV absorbers, for example cinnamates, octinoxate, benzophenes and benzophenone, oxybenzone, salicylates, octisalate, anthranilates, and p-aminobenzoic acid esters, water-insoluble vitamins, including water-insoluble vitamin derivatives, vitamin A, vitamin D2, vitamin D3, vitamin E, vitamin E acetate, and water-insoluble pharmaceuticals for topical application, antibiotic agents, antifungal agents, antibacterial agents, analgesic agents, and anti-inflammatory agents.

The hydrophobic material may be present in any amount. Preferably, it is present in an amount of about 0.2% to about 20% by weight of the total skin care composition or about 4% to about 10% by total weight of the skin care composition.

The hydrophobic material may be present in the skin care composition in a non-emulsion form relative to the acrylate copolymer; it may be present or substantially present in the form of a non-emulsified or substantially non-emulsified droplet or dispersion. Accordingly, the skin care composition itself may be referred to as a substantially non-emulsified composition. It may be preferred that at least about 80%, about 85% about 90% about 95% and about 98% of the hydrophobic material is present as non-emulsified droplets or dispersions. The droplets or dispersions may be homogenous or they may be mixtures or emulsifications of other hydrophobic or hydrophilic materials.

It may be preferred that the droplets/dispersions have an average diameter of about 0.01 to about 100 microns, about 0.1 to about 10 microns, or about 1 micron to about 50 microns.

The composition has a yield value of at least about 4. In some instances the compositions may have a yield value of about 5 to about 10 or about 4 to about 25. Yield values are measured using a Brookfield Yield Rheometer YR-1 at ambient temperature, using the appropriate spindle, e.g., spindle 72.

The composition may also include a surfactant or mixture of surfactants. The base composition may contain a surfactant. Any surfactant suitable for a skin cleansing product may be incorporated. Suitable surfactants may include a surfactant or mixture of surfactants having a combined HLB value which is at least about one HLB unit higher than the HLB required for optimal emulsion of the hydrophobic material used in the skin care composition. It may be preferred that the HLB is at least about two HLB units higher than the HLB required for optimal emulsion. The HLB of the surfactant/surfactant may be, for example, at least about 10, at least about 12, or at least about 14.

Surfactants may include amphoteric, sulfate, zwitterionic, sulfonate, anionic surfactants, such as, for example, lauryl sulfates, lauryl ether sulfates, sodium lauryl sulfate, sodium laureth sulfate, sodium methyl-2 sulfolaurate, disodium 2-sulfolaurate, sodium lauryl sulfoacetate, disodium laureth sulfosuccinate, amphoteric acetates, sodium laurylamphoacetate, betaine derivatives, cocoamidopropyl betaine, alkyl amines, alkyl imidazolines, cetylpyridinium chloride, PEG-50 stearamine, and dimethyl palmitamine.

One or more surfactants may be included in the compositions. If so, it may be preferred that the selected surfactants are sulfate surfactant(s) and amphoteric surfactant(s), preferably present in a weight ratio of about 2:1 to about 4:1 or about 3:1.

The surfactant(s) may be present in the skin care composition in any amount. For example, amounts of about 4% to 20% by weight of the total composition or about 12% to about 18% of the total composition may be present. Alternatively, water may be present in amounts of at least about 90% by weight or at least about 95% by wveight of the total composition.

Water may also be included in the composition. It may be present in an amount, for example, of at least 40% by weight of the total composition. Alternatively, the composition may contain water in an amount of at least about 10%, at least about 20%, at least about 30%, at least about 50%, at least about 60%, or at least about 70%, each by weight of the total composition.

The formulation may optionally further comprise a viscosity modifier in addition to the acrylate co-polymer, for example a cationic polymer. Any may be used—examples include chitosan, vegetable or marine colloids, and starches. If included, the skin care composition may contain about 0.1% and about 2% by weight of the cationic polymer, based on the total weight of the skin care composition; preferably, at least about 0.4%, or about 0.4% to about 1.5%.

The skin care compositions described herein may also contain any additional additives as are desired. Such additives may include insoluble particles, such as beads, polyethylene beads, encapsulates, shea butter in gelatin encapsulate, preservatives, chelators, ethylenediaminetetraacetic acid (EDTA), antibacterial agents, 1,3-dimethylol-5,5-dimethyl hydantoin (DMDMH), triclosan, or trichlocarbon, anti-oxidant agents, tocopheryl acetate, perfume, coloring agents, blocking sunscreen agents, vitamins, creatine or retinoic acid. Other additives mar include antimycotic agents, anti-inflammatory agents, menthol, adducts of an oil.

The skin care compositions described herein may have a viscosity of about 2500 to about 25,000 cps, preferably about 3500 to about 15,000 cps, as measured using a Brookfield DV-II+ viscometer, with settings at spindle: 5, speed: 20 rpm, time: 60 sec.

It has been found that the skin care compositions described herein provide useful means for delivering dermatologically beneficial compounds to the skin. For example, the skin care composition can be formulated to contain a topically active or protective compound, e.g., a sunscreen compound or vitamin, even when the active or protective compound is highly hydrophobic. The composition may be used to deliver the hydrophobic material and/or an additional, active compound to the skin surface. Specifically, one aspect of the invention includes a method of depositing a hydrophobic material on the epidermal surface. Such method includes applying to an epidermal surface any one of the compositions described herein, and subsequently removing the composition from the epidermal surface, such that at least a portion of the hydrophobic material and/or any additional, active compound(s), remain deposited on the epidermal surface. Removal of the composition can be accomplished by any means, including rinsing (with water or other fluid), wiping, blotting, scraping, evaporating and/or brushing.

The skin care compositions of the invention may be prepared by any process or procedures known or to be developed in the art. An exemplary process may include pre-mixing (e.g., by shear mixing or other means of incorporation) the selected hydrophobic material(s) and the acrylate polymer and, if desired, a surfactant in an aqueous solution to obtain a homogeneous suspension of droplets of hydrophobic material. If desired, the pH may be adjusted to obtain the desired viscosity or other properties of the formulation. At least this mixing step may be carried out at cold process temperatures described above. Any other additives or excipients may be mixed in subsequently. Preferably, where there are multiple hydrophobic materials, these components can be pre-mixed before addition to the aqueous solution.

In one aspect of the invention, a method for incorporating one or more hydrophobic materials into a skin care composition is described. The method includes forming a base, wherein the base includes an acrylate polymer and, optionally, a surfactant. This base in then combined with a hydrophobic material and a cationic polymer. The cationic polymer may be a modified guar gum or other gum, such as a salt of guar hydroxypropyltrimonium, hydroxypropyl guar and/or C₁₈ to C₁₂ alkyl hydroxypropyl guar. The salt of guar hydroxypropyltrimonium may be, e.g., any containing a suitable anion, such as guar hydroxypropyltrimonium chloride.

The method may be adapted or expanded to include preparation of any of the compositions described herein and may further be carried out as a cold process, as defined herein.

### EXAMPLES

### Example 1

### Preparation of a Moisturizing Protectant Body Wash

A skin care composition that was a body wash was prepared by mixing the first three ingredients of Table I, below, together to form a base. To that base was added the hydrogenated polydecene, and the remaining ingredients. The resulting composition was substantially non-emulsified.

TABLE I

| Ingredient | Percentage (wt %) |
|---|---|
| Water | q.s. |
| Carbopol Aqua SF-1 (30% solution) | 6.0 |
| Sodium Laureth Sulfate (25.5% | 34 |
| solution) | |
| Sodium Hydroxide (50% solution) | 0.7 |
| Cocoamidopropyl betaine (30% | 6.0 |
| solution) | |
| DMDMH (54% solution) | 0.4 |
| EDTA (40% solution) | 2.3920 |
| Hydrogenated Polydecene (25% | 4.0 |
| solution) | |
| Guar hydroxypropyltrimonium | 0.5 |
| chloride | |
| Octinoxate | 4.0 |
| Mild PCL | 5.0 |
| Citric acid (50% solution) | 0.5 |

### Example 2

### Deposition of Vitamin E on Skin Surface

A body wash composition was prepared as described in Example 1, above, except it contained vitamin E, sunflower oil and petrolatum as shown below in Table III. The control composition was a commercial body was that contained neither acrylate copolymer nor cationic polymer, and had the formulation shown in Table II, below.

TABLE II

| | |
|---|---|
| Demineralized Water | 48.70771 |
| DMDM Hydantoin | 0.41069 |
| Polyquaternium-7 | 2.568 |
| Sodium Laureth Sulfate | 33.0489 |
| (25.5%) | |
| Cocoamidopropyl betaine | 10.2764 |
| (30%) | |
| Decyl Glucoside (50%) | 2.3122 |
| EDTA (39%) | 0.2055 |
| Glycerin | 0.2 |
| Euperlan PK300 | 1.0 |
| Fragrance | 1.17 |
| Tocopheryl Acetate | 0.10006 |

TABLE III

| | Mean ug Vitamin E | |
|---|---|---|
| | Acetate/cm² skin | |
| Test Compositions | Post Wash - 1 | Post Wash - 4 |
| 0.1% Vitamin E Acetate + 4% | 0.406 | 0.728 |
| Sunflower Oil + 2% Petrolatum | | |
| 0.1% Vitamin E Acetate + 4% | 0.258 | 0.228 |
| Puresyn 6 (F) | | |
| CONTROL + 0.1% Vitamin E | 0.026 | 0.102 |
| Acetate (H) | | |

### Example 3

### Use of Yield—Increasing Polymer to Increase Yield Value of Formula

A base composition containing a base structure of 35 wt % of sodium laureth sulfate, 5 wt % of cocoamidopropyl betaine, 8.5 wt % of an acrylate copolymer (Aqua SF-1), and water (remaining content) was produced in accordance with Example 1. With the base composition, test compositions containing guar hydroxypropyl trimonium chloride (CESMETIC DP4), in the wt % concentrations of 0.2, 0.4, 0.6, 0.8 and 1.0% were produced. Viscosity and yield value were measured using a Brookfield Yield Rheometer YR-1, spindle 72.

TABLE IV

| % Guar | | |
|---|---|---|
| hydroxypropyltrimonium | | |
| Chloride by weight | Viscosity (cps) | Yield value (Pa) |
| 0.0 | 3220 | 4.02 |
| 0.2 | 4540 | 5.45 |
| 0.4 | 6160 | 7.38 |
| 0.8 | 12120 | 17.04 |
| 0.8 | >20000 | >20 |
| 1.0 | >20000 | >20 |

The results demonstrate that the addition of small quantities of a yield-increasing polymer, e.g., guar hydroxypropyl trimonium chloride, into the acrylate co-polymer structured base significantly improves structural properties of this formulation. In addition, the elastic modulus and the viscous modulus increase upon addition of the cationic polymer, and the yield stress value, which is a good measure of the suspending capabilities of the system, also increases with the addition. The effect is maximized when the concentration of the cationic polymer is greater than 0.4 wt %. The Brookfield viscosity improves upon adding the yield-increasing polymer to the system so that targeted product viscosity can be achieved without the further addition of salt.

We claim:

1. A skin care composition comprising:
a. an acrylate polymer;
b. a yield value-increasing polymer; and
c. a non-silicone hydrophobic material;
wherein the composition has a yield value of at least about 4.

2. The composition of claim 1, wherein the yield-value increasing polymer is a salt of guar hydroxypropyltrimonium.

3. The composition of claim 1, wherein the acrylate polymer is selected from a polymer of acrylic acid, a polymer of methacrylic acid, ethylacrylate, methylacryalate, polyalkenyl ethers of sucrose or polyalchols, trimethylolpropane tri(meth)acrylate, glycidyl methacrylate, and N-methylolacryamide.

4. The composition of claim 1, wherein the hydrophobic material is a semisolid hydrocarbon.

5. The composition of claim 1, wherein the hydrophobic material is selected from an oil, petrolatum, hydrogenated polydecene, a vitamin, a vegetable oil, a mineral oil and mixtures thereof.

6. The composition of claim 1, wherein the hydrophobic material is selected from sunflower oil, petrolatum, a sunscreen, an agent coated with organic material, a synthetic oil, an insect repellent, a substantially hydrophobic material, and mixtures thereof.

7. The composition of claim 1 having a yield value of about 7 to about 15.

8. The composition of claim 1, having a yield value of about 5 to about 35.

9. The composition of claim 1, having a yield value of about 4 to 50.

10. The composition of claim 1, wherein the hydrophobic material is present as a substantially homogenous dispersion.

11. The composition of claim 1, further comprising at least 4% by weight of a surfactant.

12. The composition of claim 1 further comprising an agent selected from a foam booster, a thickener, a perfume, a UV absorber, a pearlescent material, a sensate and an adduct of a vegetable oil and maleic anhydride.

13. The composition of claim 1, wherein the composition is substantially non-emulsified.

14. A method for incorporating a hydrophobic material into a skin care composition, comprising:
a. forming a base, the base comprising an acrylate polymer; and
b. combining the base with a hydrophobic material and a cationic yield value-increasing polymer.
wherein the method is carried out at a temperature of no greater than 40° C.

15. The method of claim 14, wherein the cationic polymer is a modified guar gum.

16. The method of claim 14, wherein the cationic polymer is guar hydroxypropyltrimonium chloride.

17. The method of claim 14, wherein the acrylate polymer is selected from a polymer of acrylic acid, a polymer of methacrylic acid, ethylacrylate, methylacryalate, polyalkenyl ethers of sucrose or polyalchols, trimethylolpropane tri(meth)acrylate, glycidyl methacrylate, and N-methylolacryamide.

18. The method of claim 14, wherein the hydrophobic material is selected from an oil, petrolatum, hydrogenated polydecene, a vitamin, a vegetable oil, a mineral oil, and mixtures thereof.

19. A method of depositing a hydrophobic material on an epidermal surface, the method comprising:
a. applying to the epidermal surface a composition comprising skin care composition comprising: an acrylate polymer; a modified guar gum; and a hydrophobic material, wherein the composition has a yield stress value of at least about 4; and
b. removing the composition from the epidermal surface, wherein at least a portion of the hydrophobic material remains deposition on the surface.

20. The method of claim 19, wherein the removal in accomplished by an action selected from rinsing, wiping, blotting, evaporating, and brushing.

21. The method of claim 19, wherein the acrylate polymer is selected from a polymer of acrylic acid, a polymer of methacrylic acid, ethylacrylate, methylacryalate, polyalkenyl ethers of sucrose or polyalchols, trimethylolpropane tri(meth)acrylate, glycidyl methacrylate, and N-methylolacryamide.

22. The method of claim 19, wherein the is selected from an oil, petrolatum, hydrogenated polydecene, a vitamin, a vegetable oil, a mineral oil, sunflower oil, petrolatum, and mixtures thereof.
